(19) United States
(12) Patent Application Publication
(10) Pub. No.: US 2026/0036518 A1
(43) Pub. Date: Feb. 5, 2026

(54) MICRO-ORGANISM GROWTH DETECTION METHOD, MICRO-ORGANISM ACQUISITION METHOD, MICRO-ORGANISM GROWTH DETECTION KIT, MICRO-ORGANISM ACQUISITION KIT, AND MICRO-ORGANISM GROWTH EVALUATION METHOD

(71) Applicant: National Institute of Advanced Industrial Science and Technology, Chiyoda-ku, Tokyo (JP)

(72) Inventors: Akira SASAKI, Tsukuba-shi, Ibaraki (JP); Yuri OTA, Tsukuba-shi, ibaraki (JP); Naohiro NODA, Tsukuba-shi,Ibaraki (JP); Akiko YOKOTA, Tsukuba-shi, Ibaraki (JP); Tetsushi SUYAMA, Tsukuba-shi, Ibaraki (JP)

(21) Appl. No.: 18/998,387

(22) PCT Filed: Sep. 29, 2023
(86) PCT No.: PCT/JP2023/035725

§ 371 Date: Jan. 24, 2025

(30) Foreign Application Priority Data
Jul. 29, 2022 (JP) 2022-121246

(51) Int. Cl. G01N 21/64; C12Q 1/02

(52) U.S. Cl. CPC G01N 21/6428; C12Q 1/02; G01N 2021/6439

(57) ABSTRACT

A method of detecting growth of a microorganism includes: preparing, in a W/O emulsion, a droplet containing a microorganism and a dye whose fluorescence intensity changes due to interaction with a membrane component of the microorganism; culturing the microorganism in the droplet; and detecting growth of the microorganism based on the fluorescence intensity of the dye.

### TECHNICAL FIELD

The present disclosure relates to a method of detecting growth of a microorganism, a method of obtaining a microorganism, a kit for detecting growth of a microorganism, a kit for obtaining a microorganism, and a method of evaluating growth of a microorganism.

### BACKGROUND ART

As a method of separating or culturing microorganisms, a method using a water-in-oil (W/O) emulsion has been known. In this method, droplets (culture medium) are dispersed in the oil phase, and each droplet is used as one culture site to culture a microorganism. By using a microchannel in a method of forming the droplets, hundreds of thousands or millions of droplets can be formed in several minutes to enable a high-throughput process. Further, the number of microorganisms present in the aqueous phase and the number of droplets may be controlled to form droplets in each of which one microbial cell is encapsulated, and this allows culturing from the one cell.

In cases where droplets are used to culture microorganisms, the number of cells per droplet follows a Poisson distribution. As the ratio of droplets in each of which only one cell is encapsulated increases among the droplets in which one or more cells are encapsulated, the ratio of droplets generated without inclusion of any microorganism also increases. In this case, if droplets showing growth of microorganisms can be selectively collected, they can be used, for example, for analysis and scale-up of each microorganism.

Detection of the droplets in which microorganisms are present has been carried out by a method in which autofluorescence of the microbial cells are detected, and a method that uses a reagent that reacts with an extracellular secretion. For example, Patent Literature 1 discloses use of a fluorescence resonance energy transfer (FRET)-type fluorescence-modified nucleic acid probe for detection of droplets in which microorganisms are present. The fluorescence-modified nucleic acid probe has a fluorescent group and a quenching group at the 5′-end and the 3′-end, respectively. In the droplets in which microorganisms are present, cleavage of the fluorescence-modified nucleic acid probe by RNase secreted from the microorganisms eliminates the FRET, resulting in an increase in the fluorescence intensity.

### CITATION LIST

### Patent Literature

Patent Literature 1: International Publication No. WO 2019/073902

### SUMMARY OF INVENTION

### Technical Problem

In the method based on the detection of the autofluorescence of cells, low sensitivity has led to difficulty in accurate detection of the droplets in which microorganisms have grown. In the method using the fluorescence-modified nucleic acid probe disclosed in Patent Literature 1 above, an attempt to detect microorganisms in an environmental sample has led to a false positive due to an increase in the background fluorescence value by, for example, RNase contained in impurities in the environment and a small amount of RNase contained in the culture medium, so that accurate detection of the growth of the microorganisms in the droplets has been impossible in some cases. There have also been inconveniences including the fact that a time lag occurs between the actual growth and the degradation by the RNase, and the fact that the number of cells present in the droplets does not directly correlate with the fluorescence intensity.

The present disclosure was made in view of the above circumstances, and aims to provide a method of detecting growth of a microorganism, the method being capable of accurate detection of droplets in which the microorganism has grown, a method of obtaining a microorganism, a kit for detecting growth of a microorganism, a kit for obtaining a microorganism, and a method of evaluating growth of a microorganism.

### Solution to Problem

A method of detecting growth of a microorganism according to a first aspect of the present disclosure includes:
preparing, in a W/O emulsion, a droplet containing a microorganism and a dye whose fluorescence intensity changes due to interaction with a membrane component of the microorganism;
culturing the microorganism in the droplet; and
detecting growth of the microorganism based on fluorescence intensity of the dye.

The dye may be a dye whose fluorescence intensity increases due to insertion of the dye into outer membrane of a cell of the microorganism.

The dye may be N-(3-triethylammoniumpropyl)-4-(4-(dibutylamino)styryl)pyridinium dibromide or 4-[6-[4-(diethylamino)phenyl]-1,3,5-hexatrien-1-yl]-1-[3-(triethylammonio)propyl]-pyridinium dibromide.

The microorganism may include a plurality of species.

A method of obtaining a microorganism according to a second aspect of the present disclosure includes:
preparing, in a W/O emulsion, a droplet containing a microorganism and a dye whose fluorescence intensity changes due to interaction with a membrane component of the microorganism;
culturing the microorganism in the droplet;
detecting growth of the microorganism based on fluorescence intensity of the dye; and
collecting the droplet in which the growth of the microorganism has been detected.

A kit for detecting growth of a microorganism in a droplet in a W/O emulsion according to a third aspect of the present disclosure includes:
a dye whose fluorescence intensity changes due to interaction with a membrane component of the microorganism.

A kit for obtaining a microorganism in a droplet in a W/O emulsion according to a fourth aspect of the present disclosure includes:
a dye whose fluorescence intensity changes due to interaction with a membrane component of the microorganism.

A method of evaluating growth of a microorganism according to a fifth aspect of the present disclosure includes:
using a dye whose fluorescence intensity changes due to interaction with a membrane component of the microorganism, as a reporter for growth of the microorganism in a droplet in a W/O emulsion.

### Advantageous Effects of Invention

The present disclosure enables accurate detection of droplets in which microorganisms have grown.

### BRIEF DESCRIPTION OF DRAWINGS

FIG. 1 is a diagram illustrating a microscopic image of droplets after the culture in Test Example 1; each bar in the figure corresponds to 100 μm;

FIG. 2A is a diagram illustrating the fluorescence intensity of droplets containing no cell-membrane-staining reagent in Test Example 2;

FIG. 2B is a diagram illustrating the fluorescence intensities of droplets containing a cell-membrane-staining reagent (FM1-43) in Test Example 2;

FIG. 2C is a diagram illustrating the fluorescence intensities of droplets containing a cell-membrane-staining reagent (FM4-64) in Test Example 2;

FIG. 3 is a diagram illustrating a microscopic image of droplets after the culture in Test Example 3; each bar in the figure corresponds to 100 82 m;

FIG. 4 is a diagram illustrating the fluorescence intensities of droplets containing a dye that emits green fluorescence in Test Example 4; and

FIG. 5 is a diagram illustrating the fluorescence intensities of droplets containing a dye that emits red fluorescence in Test Example 4.

### DESCRIPTION OF EMBODIMENTS

Embodiments according to the present disclosure are described with

reference to drawings. The present disclosure is not limited by the following embodiments.

### Method of Detecting Growth of Microorganism

The method of detecting growth of a microorganism according to the present embodiment includes:
preparing, in a W/O emulsion, a droplet containing a microorganism and a dye whose fluorescence intensity changes due to interaction with a membrane component of the microorganism (Step 1);
culturing the microorganism in the droplet (Step 2); and
detecting growth of the microorganism based on fluorescence intensity of the dye (Step 3).
The W/O emulsion means a state where droplets (water droplets) in the form of fine particles are present as a dispersed phase in an oil phase that is a continuous phase. A “droplet” is a compartmentalized drop of water in an emulsion. The aqueous phase constituting the W/O emulsion is not limited as long as it is a hydrophilic liquid that is immiscible with the oil phase. Examples of the liquid that can be used as the aqueous phase include water, lake water, and seawater. Since microorganisms are cultured in the droplets, the liquid is preferably a medium that is immiscible with the oil phase, such as, for example, LB medium or R2A medium.

The aqueous phase for the droplets prepared in Step 1 contains a microorganism and the dye. The microorganism is not particularly limited as long as it is capable of growing in the droplets. The microorganism may be either a prokaryote or a eukaryote. Examples of the microorganism include Escherichia coli, Bacillus subtilis, actinomycetes, and yeast.

The method of detecting growth of a microorganism according to the present embodiment is applicable to environmental samples such as air, lake water, seawater, and soil. Thus, the droplets may contain microorganisms contained in an environmental sample, and the microorganisms may therefore be a plurality of species. Each microorganism may also be an artificially prepared microorganism, such as a recombinant microorganism having a gene introduced therein.

The dye is a dye whose fluorescence intensity changes, preferably increases, due to interaction with a membrane component of a microorganism in the droplet. The dye is preferably a dye whose fluorescence intensity increases due to insertion of the dye into the outer membrane of a cell of the microorganism. Examples of such a dye include styryl dyes. Styryl dyes are also called Fei Mao dyes, and examples of those dyes include N-(3-triethylammoniumpropyl)-4-(4-(dibutylamino)styryl)pyridinium dibromide; FM (trademark, the same applies hereafter) 1-43), 4-[2-[4-(dipentylamino)phenyl]ethenyl]-1-[3-(triethylaminio)propyl]pyridinium bromide (FM1-84), N-(3-triethylammoniumpropyl)-4-(4-(diethylamino)styryl)pyridinium dibromide; FM2-10), 4-[6-[4-(diethylamino)phenyl]-1,3,5-hexatrien-1-yl]-1-[3-(triethylammonio)propyl]-pyridinium dibromide (FM4-64), and N-(3-trimethylammoniumpropyl)-4-(6-(4-(diethylamino)phenyl)hexatrienyl)pyridinium dibromide (FM5-95). N-(3-triethylammoniumpropyl)-4-(4-(dibutylamino) styryl) pyridinium dibromide and 4-[6-[4-(diethylamino) phenyl]-1,3,5-hexatrien-1-yl]-1-[3-(triethylammonio)propyl]-pyridinium dibromide are especially preferred as the dye.

The oil phase constituting the W/O emulsion is not particularly limited as long as it is a hydrophobic liquid that is immiscible with the aqueous phase. Examples of such an oil phase include FC40, Novec (trademark, the same applies hereafter) 7500, mineral oil, and combinations thereof.

For stabilization of the W/O emulsion, a surfactant is added to at least one of the aqueous phase and the oil phase. Examples of the surfactant used for the aqueous phase include Span (trademark) 80 and Tween (trademark) 20. Examples of the surfactant for the oil phase include 008-FluoroSurfactant, Pico-surf (trademark) 1, and Krytox (trademark). The concentration of the surfactant added to the aqueous phase or the oil phase can be appropriately adjusted according to conditions such as the type of surfactant used and the size of the droplets.

The W/O emulsion may be any W/O emulsion as long as it allows growth of the microorganism in the droplets and detection of the growth of the microorganism. The size of each droplet is not particularly limited as long as it allows growth of the microorganism and detection of the growth of the microorganism. The droplet has a diameter of, for example, 5 to 500 μm, 10 to 300 μm, 15 to 200 μm, or 20 to 150 μm.

Methods of preparing W/O emulsions are known. W/O emulsions can be prepared, for example, using commercially available apparatuses such as an On-chip Droplet Generator (manufactured by On-chip Biotechnologies), a Droplet Generator (manufactured by Bio-Rad), and QX100 (manufactured by Bio-RAD).

Regarding Step 2, the method of culturing a microorganism in a droplet in a W/O emulsion is known. An apparatus required for the culture such as on a microchannel may be appropriately selected to culture the microorganism. The culture conditions may be any condition as long as the growth of the microorganism contained in the droplet is possible. Preferred culture conditions may be appropriately set in accordance with the purpose of the culture or the microorganism to be cultured. The temperature condition for the culture is, for example, 4 to 95° C. The culture period may be, for example, several hours, several days, or several months, or the culture may be carried out over a period of not less than several days to not less than several months.

In Step 3, the growth of the microorganism is detected based on the fluorescence intensity of the dye described above. The fluorescence intensity of the dye can be measured with a commercially available apparatus. Since the fluorescence intensity of the dye changes due to its interaction with a membrane component of the microorganism, the change in the fluorescence intensity can be detected by comparison with the fluorescence intensity of a droplet before the beginning of the culture or a droplet not containing the microorganism. In cases where a dye whose fluorescence intensity increases due to interaction with a membrane component of the microorganism is used, an increase in the microorganism by its growth leads to an increase in the dye exposed to the membrane component, and hence to an increase in the dye that interacts with the membrane component. Therefore, the fluorescence intensity increases in proportion to the number of cells of the microorganism in the droplet.

In the method of detecting growth of a microorganism according to the present embodiment, the growth of the microorganism is detected based on the fluorescence intensity of the dye whose fluorescence intensity changes due to interaction with a membrane component of the microorganism. This change in the fluorescence intensity enables highly sensitive measurement compared to cases where the dye has not been allowed to act on the cells, so that droplets in which the microorganism has grown can be accurately detected. Further, since the method of detecting growth of a microorganism according to the present embodiment is based on the detection of a change in the fluorescence intensity due to an increase in the dye that interacts with a membrane component, the method is less susceptible to effects of components other than those of the microorganism contained in the environmental sample and the culture medium, so that an increase in the measured background value can be avoided. Therefore, the growth of the microorganism can be accurately detected. Further, since the change in the fluorescence intensity due to an increase in the dye that interacts with a membrane component shows direct correlation with the actual growth of the microorganism, the growth of the microorganism can be detected with no time lag.

According to the method of detecting growth of a microorganism according to the present embodiment, in cases where microorganisms of a plurality of species are encapsulated and cultured in one droplet, effects of interactions between the microorganisms encapsulated in the droplet on the growth ability can be studied.

Further, by encapsulating one microbial cell per droplet, droplets containing microorganisms that exhibit different growth abilities can be obtained. In particular, for the purpose of isolation of a particular microorganism from an environmental sample containing microorganisms of a plurality of species, each droplet preferably contains only one microbial cell. Methods of preparing a W/O emulsion such that each droplet contains only one microbial cell are known. For example, droplets each containing only one microbial cell can be prepared by controlling the number of microorganisms present in the aqueous phase and the number of droplets according to a Poisson distribution.

Further, in cases where a plurality of cells is encapsulated in each droplet, the culture period required for the growth to a detectable cell number can be reduced, which is preferred. Further, in cases where microorganisms having a symbiotic relationship with each other are encapsulated in the same droplet, growth-promoting effect can be expected due to, for example, interactions between the microorganisms.

Step 3 may be carried out on a microchannel. By combination of a microchannel with a commercially available cell sorter or the like, high-throughput measurement of the fluorescence intensity is possible.

### Method of Obtaining Microorganism

The method of obtaining a microorganism according to the present embodiment includes, in addition to the above-described Steps 1, 2, and 3, collecting the droplets in which growth of the microorganism has been detected (Step 4). Methods of collecting or separating droplets in which growth of a microorganism has been detected, in other words, in which the fluorescence intensity of a dye has changed after culture, are known. For example, a commercially available chip-type cell sorter can be used to perform sorting of droplets in which the fluorescence intensity has changed. In cases where Step 4 is carried out on a microchannel, the microchannel can be combined with a commercially available cell sorter to enable high-throughput measurement of the fluorescence intensity, and separation of droplets according to the detection result.

By the method of obtaining a microorganism according to the present embodiment, droplets in which the microorganism has grown can be accurately detected, so that the desired microorganism can be more securely obtained. Further, for an environmental sample containing microorganisms of a plurality of species, encapsulation of only one microbial cell per droplet enables detection of the growth in accordance with the properties of each microorganism contained in each droplet, so that droplets in which different growth abilities have been found can be isolated or separated individually. Since the droplets in which different growth abilities are found can be selectively and individually separated, analysis of each microorganism and scaling up of the culture are possible.

### Kit for Detecting Growth of Microorganism and Kit for Obtaining Microorganism

In another embodiment, a kit for detecting growth of a microorganism in a droplet in a W/O emulsion is provided. The kit for detecting growth of a microorganism includes the dye described above. Further, the kit for detecting growth of a microorganism may include: an aqueous phase of the W/O emulsion, such as a medium; an oil phase of the W/O emulsion; and a surfactant for stabilization of the W/O emulsion. The kit for detecting growth of a microorganism may include, for example, a microchannel for use in culture or the like, and a culture dish. In another embodiment, the kit for detecting growth of a microorganism can be used as a kit for obtaining a microorganism in a droplet in a W/O emulsion.

### Method of Evaluating Growth of Microorganism

In another embodiment, a method for evaluating growth of a microorganism in a droplet in a W/O emulsion is provided. The method of evaluating growth of a microorganism includes using a dye whose fluorescence intensity changes due to interaction with a membrane component of the microorganism, as a reporter for growth of the microorganism in a droplet in a W/O emulsion. The reporter for growth of the microorganism is an indicator of the growth of the microorganism in the droplet. In cases where the dye employed is a dye whose fluorescence intensity increases due to insertion of the dye into the outer membrane of a cell of the microorganism, the fluorescence intensity of the dye in the droplet correlates with the number of microbial cells in the droplet. Therefore, the number of microbial cells in the droplet can be evaluated based on the fluorescence intensity of the dye. Specifically, for example, a calibration curve may be prepared in advance based on the number of microbial cells and the fluorescence intensity of the dye corresponding to the number of the cells, and the calibration curve may be used for an evaluation sample to determine the number of microbial cells in a droplet based on the fluorescence intensity of the dye in the droplet.

### EXAMPLES

The present disclosure is described more concretely by way of the following Examples. However, the present disclosure is not limited by Examples.

### Test Example 1: Detection of E. coli in W/O Droplets

A study was carried out to determine whether the growth of Escherichia coli (E. coli) in W/O droplets can be detected by measuring the fluorescence of a cell-membrane-staining reagent. The following Samples 1 to 3 were used as aqueous phases.

Sample 1: LB medium containing E. coli

Sample 2: LB medium containing the cell-membrane-staining reagent FM1-43 (manufactured by ThermoFisher Scientific) at 10 μM and E. coli

Sample 3: LB medium containing the cell-membrane-staining reagent FM4-64 (manufactured by ThermoFisher Scientific) at 10 μM and E. coli

Novec 7500 (manufactured by On-chip Biotechnologies) containing 2% of 008-FluoroSurfactant was used as an oil phase. Droplets with a diameter of about 30 μm were prepared using an On-chip Droplet Generator as a W/O droplet preparation apparatus. The concentration of E. coli in the aqueous phase was adjusted to 0.1 cell in terms of the average number of cells contained per droplet (0.1 cell/droplet). The E. coli was subjected to static culture in the prepared droplets overnight at 37° C.

### Results

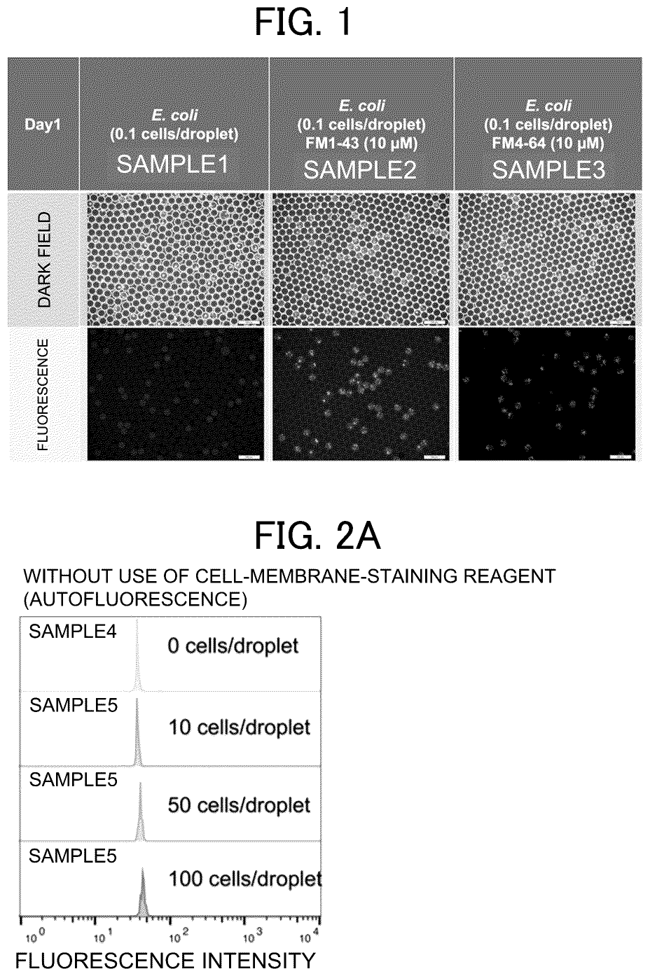

As a result of microscopic observation of droplets after the culture, droplets in which E. coli had grown showed strong fluorescence in Samples 2 and 3, which contained the cell-membrane-staining reagent, as illustrated in FIG. 1. It was thus shown that measurement of the fluorescence of the cell-membrane-staining reagent enables detection of growth of E. coli in W/O droplets.

### Test Example 2: Study of Number of E. coli Cells Detectable with Cell-Membrane-Staining Reagents

The intensities of cell-membrane-staining reagents were compared with the intensity of autofluorescence, to study whether use of the cell-membrane-staining reagents improves the detection sensitivity. The following Samples 4 to 9 were used as aqueous phases.
Sample 4: LB medium
Sample 5: LB medium containing E. coli
Sample 6: LB medium containing 10 μM FM1-43
Sample 7: LB medium containing 10 μM FM1-43 and E. coli
Sample 8: LB medium containing 10 μM FM4-64
Sample 9: LB medium containing 10 μM FM4-64 and E. coli

Novec 7500 containing 2% of 008-FluoroSurfactant was used as an oil phase to prepare droplets with a diameter of about 30 μm as in Test Example 1. The concentrations of E. coli in Samples 5, 7, and 9 were adjusted to 10 cells/droplet, 50 cells/droplet, and 100 cells/droplet, respectively. The prepared droplets were subjected to static culture overnight at 37° C. The fluorescence intensity was measured using On-chip Sort (manufactured by On-chip Biotechnologies) immediately after the preparation of the droplets.

### Results

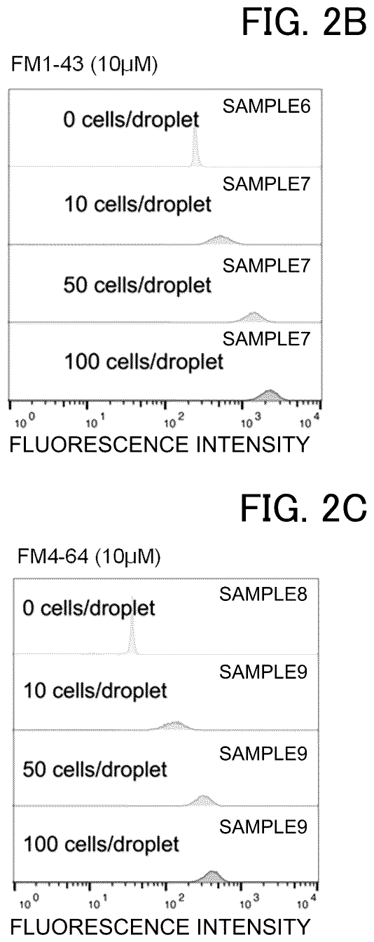

As illustrated in FIG. 2A, in droplets containing no cell-membrane-staining reagent, differences in the number of cells of E. coli could not be identified based on the fluorescence intensity of autofluorescence. On the other hand, in the droplets containing FM1-43 and FM4-64 illustrated in FIGS. 2B and 2C, respectively, the fluorescence intensity increased as the number of E. coli cells increased, and increases in the fluorescence intensity in accordance with the number of cells could be detected in droplets containing not less than 10 E. coli cells.

### Test Example 3: Detection of Environmental Microorganisms Using Cell-Membrane-Staining Reagents

A test was carried out to determine whether cell growth can be detected using cell-membrane-staining reagents when various types of microorganisms contained in the soil are cultured. LB medium containing 10 μM FM1-43 and soil microorganisms, and LB medium containing 10 μM FM4-64 and soil microorganisms were used as aqueous phases. Novec 7500 containing 2% of 008-FluoroSurfactant was used as an oil phase. Droplets with a diameter of about 100 μm were prepared using an On-chip Droplet Generator as a water-in-oil droplet preparation apparatus. In the prepared droplets, the soil microorganisms were subjected to static culture overnight at 25° C.

### Results

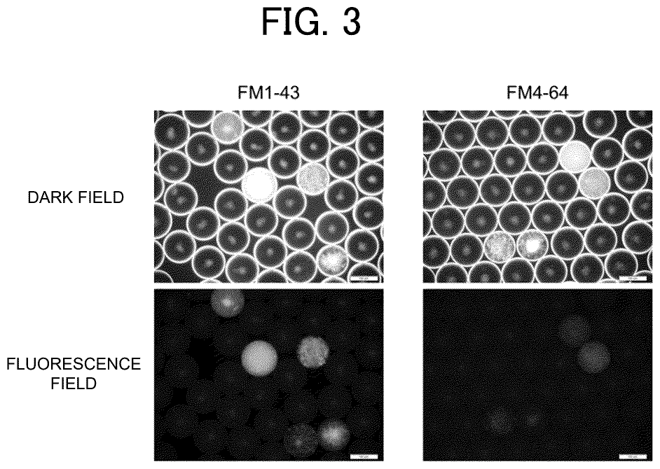

FIG. 3 illustrates images of droplets observed under the microscope. In both samples, under the microscope, increased fluorescence was found in droplets in which the soil microorganisms had grown.

### Test Example 4: Detection of Oleaginous Yeast Using Cell-Membrane-Staining Reagents

A study was carried out to determine whether cell growth of an oleaginous yeast (Lipomyces starkeyi), which is a eukaryote, can be detected. As aqueous phases, PBS suspensions containing the oleaginous yeast (100 cells/droplet) and various staining reagents (10 μM FM1-43 (designated as Dye #1), Cell Navigator (trademark, the same applies hereinafter) Green (manufactured by AAT)×500 dilution (designated as Dye #2), PlasMem Bright Green (manufactured by Dojindo)×200 dilution (designated as Dye #3), μM FM4-64 (designated as Dye #4), CellMask (trademark, the same applies hereafter) 10 Orange (manufactured by Invitrogen)×1000 dilution (designated as Dye #5), or 20 82 M MemGlow (trademark, the same applies hereafter) 560 (manufactured by Funakoshi) (designated as Dye #6)) were used. For comparison, a sample without the oleaginous yeast was used. Dye #6 is a dye whose fluorescence is suppressed to a minimal level by formation of self-quenching nanoparticles before binding to the lipid bilayer, but whose fluorescence intensity increases due to interaction with the lipid bilayer.

Novec 7500 containing 2% of 008-FluoroSurfactant was used as an oil phase. Droplets with a diameter of about 120 μm were prepared using a Droplet Generator as a water-in-oil droplet preparation apparatus. The fluorescence intensity was measured for each droplet using On-chip Sort immediately after the preparation of the droplets.

### Results

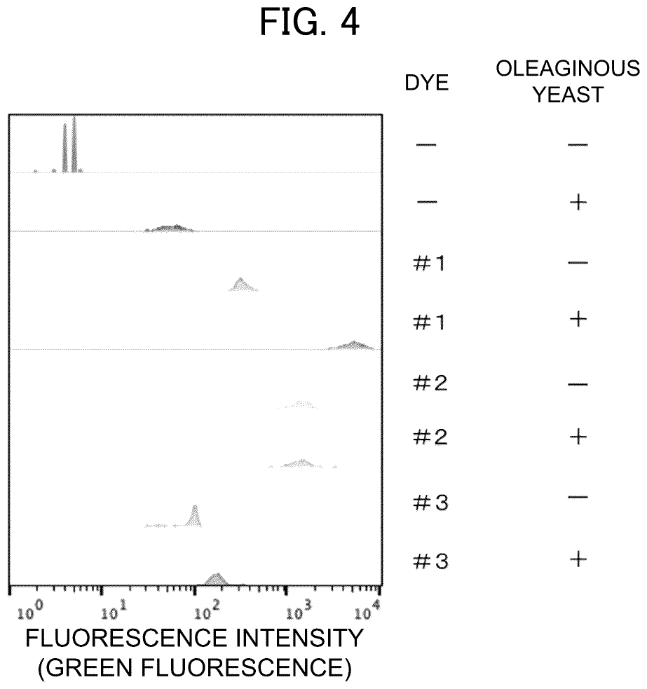

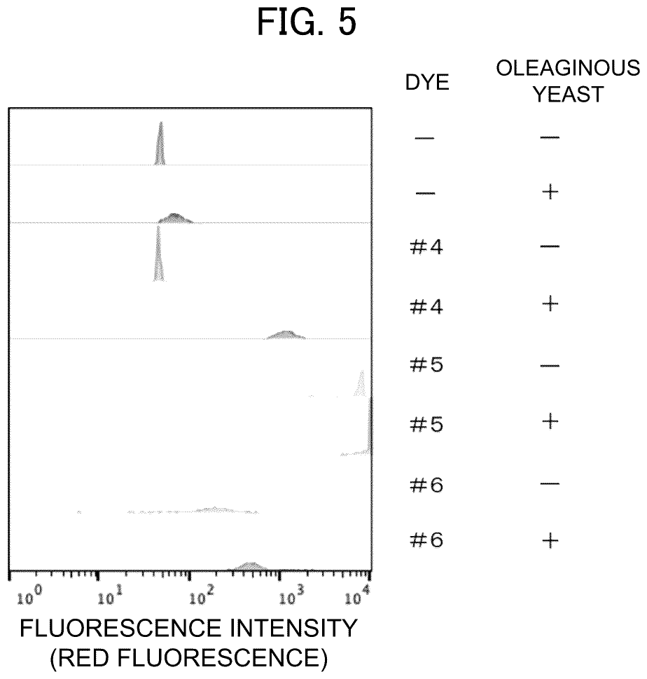

FIGS. 4 and 5 illustrate the results for the dyes that emit green fluorescence and the dyes that emit red fluorescence, respectively. In droplets containing FM1-43 (Dye #1) and FM4-64 (Dye #4), the fluorescence intensity was increased in accordance with the presence of the oleaginous yeast. On the other hand, the other dyes, #2, #3, #5, and #6, failed to clearly identify the presence of the oleaginous yeast due to, for example, insufficiency of the staining intensity itself in the droplets, or too high background.

The foregoing describes some example embodiments for explanatory purposes. Although the foregoing discussion has presented specific embodiments, persons skilled in the art will recognize that changes may be made in form and detail without departing from the broader spirit and scope of the invention. Accordingly, the specification and drawings are to be regarded in an illustrative rather than a restrictive sense. This detailed description, therefore, is not to be taken in a limiting sense, and the scope of the invention is defined only by the included claims, along with the full range of equivalents to which such claims are entitled.

This application claims the benefit of Japanese Patent Application No. 2022-121246, filed on Jul. 29, 2022, the entire disclosure of which is incorporated by reference herein.

### INDUSTRIAL APPLICABILITY

The present disclosure is suitable for testing the presence or absence of microorganisms, and for separation and analysis of microorganisms.

1. A method of detecting growth of a microorganism, the method comprising:
preparing, in a W/O emulsion, a droplet containing a microorganism and a styryl dye;
culturing the microorganism in the droplet; and
detecting growth of the microorganism based on fluorescence intensity of the styryl dye.

2. The method according to claim 1, wherein the styryl dye is N-(3-triethylammoniumpropyl)-4-(4-(dibutylamino)styryl)pyridinium dibromide, 4-[2-[4-(dipentylamino)phenyl]ethenyl]-1-[3-(triethylaminio)propyl]pyridinium bromide, N-(3-triethylammoniumpropyl)-4-(4-(diethylamino)styryl)pyridinium, 4-[6-[4-(diethylamino)phenyl]-1,3,5-hexatrien-1-yl]-1-[3-(triethylammonio)propyl]-pyridinium dibromide, or N-(3-trimethylammoniumpropyl)-4-(6-(4-(diethylamino)phenyl)hexatrienyl)pyridinium dibromide.

3. The method according to claim 2, wherein the styryl dye is N-(3-triethylammoniumpropyl)-4-(4-(dibutylamino)styryl)pyridinium dibromide or 4-[6-[4-(diethylamino)phenyl]-1,3,5-hexatrien-1-yl]-1-[3-(triethylammonio)propyl]-pyridinium dibromide.

4. The method according to claim 1, wherein the microorganism includes a plurality of species.

5. A method of obtaining a microorganism, the method comprising:
preparing, in a W/O emulsion, a droplet containing a microorganism and a styryl dye;
culturing the microorganism in the droplet;
detecting growth of the microorganism based on fluorescence intensity of the styryl dye; and
collecting the droplet in which the growth of the microorganism has been detected.

6. A kit for detecting growth of a microorganism in a droplet in a W/O emulsion, the kit comprising a styryl dye.

7. A kit for obtaining a microorganism in a droplet in a W/O emulsion, the kit comprising a styryl dye.

8. A method of evaluating growth of a microorganism, the method comprising using a styryl, as a reporter for growth of the microorganism in a droplet in a W/O emulsion.
